(19) United States
(12) Patent Application Publication
(10) Pub. No.: US 2026/0345130 A1
(43) Pub. Date: Nov. 5, 2026

(54) SYSTEM AND METHODS FOR MEASUREMENT OF PARAMETERS SUCH AS INTRACRANIAL PRESSURE

(71) Applicant: Massachusetts Institute of Technology, Cambridge, MA (US)

(72) Inventors: Syed Muhammad Imaduddin, Cambridge, MA (US); Charles G. Sodini, Belmont, MA (US); Thomas Heldt, Cambridge, MA (US)

(73) Assignee: Massachusetts Institute of Technology, Cambridge, MA (US)

(21) Appl. No.: 19/117,682

(22) PCT Filed: Oct. 6, 2023
(86) PCT No.: PCT/US2023/076170

§ 371 Date: Apr. 2, 2025

Related U.S. Application Data
(60) Provisional application No. 63/413,875, filed on Oct. 6, 2022.

(51) Int. Cl. A61B 8/00; A61B 8/04; A61B 8/06; G16H 50/30

(52) U.S. Cl. CPC A61B 8/5223; A61B 8/04; A61B 8/06; G16H 50/30

(57) ABSTRACT

Systems and methods related to determination of patient parameters such as intracranial pressure (ICP), intracranial compliance (ICC), and cerebrovascular resistance (CVR) are provided. In some instances, the parameters are determined based at least in part on measures two or more parameters related to distension of a vessel and blood flow through the vessel (e.g., arterial blood pressure and cerebral blood flow). In some instances, the parameters are determined based at least in part on measurements performed using one or more probes (e.g., a single probe) at a single patient site. The systems and method described may, in some instances, facilitate determination of these parameters non-invasively and/or at a point-of-care.

### RELATED APPLICATIONS

This application claims priority under 35 U.S.C. § 119 (e) to U.S. Provisional Patent Application No. 63/413,875, filed Oct. 6, 2022, and entitled “System and Methods for Measurement of Parameters such as Intracranial Pressure,” which is incorporated herein by reference in its entirety for all purposes.

### TECHNICAL FIELD

Systems and methods related to determination of patient parameters such as intracranial pressure are generally described.

### SUMMARY

Systems and methods related to determination of patient parameters such as intracranial pressure (ICP), intracranial compliance (ICC), and cerebrovascular resistance (CVR) are provided. In some instances, the parameters are determined based at least in part on measurements performed using one or more probes (e.g., a single probe) at a single patient site. The systems and method described may, in some instances, facilitate determination of these parameters non-invasively and/or at a point-of-care.

The subject matter of the present invention involves, in some cases, interrelated products, alternative solutions to a particular problem, and/or a plurality of different uses of one or more systems and/or articles.

In one aspect, systems are provided. In some embodiments, the system comprises: at least one hardware processor; and at least one non-transitory computer-readable storage medium storing processor-executable instructions that, when executed by the at least one hardware processor, cause the at least one hardware processor to perform: obtaining a set of data identifying a measure of arterial blood pressure and a measure of volumetric cerebral blood flow of a patient; and outputting information, based at least in part on the set of data, indicating a measure of a value in absolute units of one or more of the following: intracranial pressure (ICP), intracranial compliance (ICC), and cerebrovascular resistance (CVR).

In some embodiments, the system further comprises one or more probes configured to perform measurements at a patient site and send information about the measurements to the at least one hardware processor, wherein the obtaining the set of data identifying a measure of arterial blood pressure and a measure of volumetric cerebral blood flow of a patient is based at least in part on the information about the measurements at the patient site.

In some embodiments, the measure of a value in absolute units of ICC is a measure of a value in absolute units of a regional ICC and/or wherein the measure of a value in absolute units of CVR is a measure of a value in absolute units of a regional CVR.

In some embodiments, the system comprises at least one hardware processor; one or more probes configured to perform measurements at a patient site and send information about the measurements to the at least one hardware processor; and at least one non-transitory computer-readable storage medium storing processor-executable instructions that, when executed by the at least one hardware processor, cause the at least one hardware processor to perform: obtaining a set of data, based at least in part on the information about the measurements at the patient site, identifying measures of two or more parameters related to distension of a vessel and blood flow through the vessel; and outputting information, based at least in part on the set of data, indicating a value of one or more of the following: intracranial pressure (ICP), intracranial compliance (ICC), and cerebrovascular resistance (CVR).

In some embodiments, the value indicated in the output information is a value in absolute units.

In some embodiments, the measures of two or more parameters related to distension of a vessel and blood flow through the vessel comprise one or both of: (1) a measure of arterial blood pressure; and (2) a measure of cerebral blood flow.

In some embodiments, the measure of cerebral blood flow comprises a measure of cerebral blood flow velocity and/or a measure of volumetric cerebral blood flow.

In some embodiments, the one or more probes comprise only a single probe.

In some embodiments, the one or more probes comprise an ultrasound probe.

In some embodiments, the information output by the at least one hardware processor indicates a measure of a value in absolute units of ICP.

In some embodiments, the measure of a value in absolute units of ICP is in the units of mmHg, pascals, bars, pounds per square inch (PSI), atmospheres, or a multiple thereof.

In some embodiments, the information output by the at least one hardware processor indicates a measure of a value in absolute units of ICC.

In some embodiments, the measure of a value in absolute units of ICC is in the units of mL/mmHg, mL/pascal, mL/bar, mL/PSI, mL/atmosphere, or a multiple thereof.

In some embodiments, (a) the data identifying a measure of arterial blood pressure comprise arterial blood pressure waveforms converted from vessel diameter waveforms using pre-trained models, first principle models, and/or by calibration with an arm-cuff; and/or (b) the data identifying a measure of volumetric cerebral blood flow of a patient are calculated via integration of a measure of cerebral blood flow velocity using vessel diameter waveforms; and/or (c) the data identifying a measure of arterial blood pressure and/or the data identifying a measure of cerebral blood flow are obtained based at least in part on information about measurements from a biplanar imaging sequence.

In some embodiments, the values of ICP, ICC, and/or CVR are calculated by feeding arterial blood pressure and volumetric cerebral blood flow waveforms to a modified physiologic model.

In some embodiments, the system is a point-of-care system configured to perform a continuous, model-based, non-invasive, real-time, patient-specific measurement of a measure of intracranial pressure (ICP), intracranial compliance (ICC), and/or cerebrovascular resistance (CVR).

In some embodiments, the system further comprises a display configured to communicate to at least one user the information output from the at least one hardware processor.

In some embodiments, the instructions, when executed by the at least one hardware processor, further cause the at least one hardware processor to perform: generating an alert regarding the administration of a treatment for relieving a neuropathological condition based at least in part on the information output from the at least one hardware processor; and transmitting the alert to the display such that the alert triggers the display to communicate the alert to the at least one user.

In another aspect, methods are provided. In some embodiments, the method comprises obtaining a measure of one or more of the following using the system of any of the systems described in this disclosure: intracranial pressure (ICP), intracranial compliance (ICC), and cerebrovascular resistance (CVR).

In some embodiments, the method further comprises administering a treatment for relieving a neuropathological condition to a subject based at least in part on the obtained measure.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1E shows an illustration (FIG. 1A) of measurements according to some embodiments, where an ultrasound imaging device performs (time-interleaved) B-mode (FIG. 1B) and color flow (FIG. 1C) measurements, the B-mode images are processed to detect vessel edges and therefore estimate vessel diameters (FIG. 1E), the velocity measurements from the color flow data are integrated to estimate the volumetric flow (FIG. 1D), and the diameters can be converted to arterial blood pressure (ABP) waveforms using pre-trained models or by calibrating with an arm-cuff;

FIG. 2 shows an illustration of an existing measurement setup in which radial arterial catheter (RAC) is used for ABP measurement at the wrist, while transcranial doppler (TCD) is performed at the middle cerebral artery (MCA) to estimate the cerebral blood flow velocity (CBFV);

FIG. 3A shows a diagram of an electrical analogue of an existing cerebral hemodynamics model developed;

FIG. 3B shows a diagram of a modified model, in accordance with some embodiments, incorporating the ICC, where pₐ: ABP; \acute{q}: CBF; C or C₁: vascular and brain compliance; C₂: linearized ICC; R: CVR; pᵥ: venous pressure; pᵢ: ICP; p_{dc}: DC operating point for linearized ICC; the linear capacitors form an effective compliance, Cₑ;

FIG. 4A is a block diagram of an illustrative computer system that may be used in implementing some embodiments of the technology described herein;

FIG. 4B is a block diagram of an illustrative system comprising a computing device and a probe that may be used in implementing some embodiments of the technology described herein;

FIG. 5 shows a coordinate system that can be used in some embodiments, where the imaging plane may be rotated about the z-axis by an angle ψ, coordinates within the imaging plane are given by (ρ,φ) pairs, and transducer array elements are indexed by (i,j) pairs);

FIG. 6 shows a flow chart of vessel localization process for a common carotid artery, according to some embodiments;

FIG. 7A shows a color flow region enclosed in a parallelogram oriented at an azimuth, φ, relative to the z axis, where a vessel oriented at an angle, α, relative to the x-axis is imaged and makes a net angle θ relative to the received beams labeled from 1 to 3, and the direction of blood flow is shown by the arrows pointing to the upper left, according to some embodiments;

FIG. 7B shows velocity profiles measured along each beam, according to some embodiments;

FIG. 7C, shows the profiles from FIG. 7B spatially aligned, according to some embodiments;

FIG. 7D shows the profiles from FIG. 7C averaged (lower profile) and scaled by cos⁻¹(θ) (upper profile), according to some embodiments;

FIG. 8A shows a color flow region enclosed in a parallelogram oriented at an azimuth, φ, relative to the z axis, where a vessel oriented at an angle, α, relative to the x-axis is imaged and makes a net angle θ relative to the received beams labeled from 1 to 3, and the direction of blood flow is shown by the arrows pointing to the upper left, according to some embodiments;

FIG. 8B shows the spatially-averaged, angle-corrected velocity profile as a function of distance, ρ, along the received beam, where the profile is projected to the radial axis such that the black circle at (x₁,z₁) is assigned a radial value of r₁, according to some embodiments;

FIG. 8C shows the projected velocity profile along the radial axis, according to some embodiments;

FIGS. 9A-9D show estimated angle-corrected velocity profiles for two frames, where for the overlaid B-mode and CF frames, regions are marked to indicate flow towards and away from the ultrasound transducer, respectively, the raw and spline-interpolated profiles are shown as dotted and solid lines, respectively, negative velocities have been wrapped around to positive values, and the radial coordinates are normal to the center axis of the vessel, according to some embodiments;

FIGS. 10A-10C show example data of flow (FIG. 10A), diameter (FIG. 10B), and mean velocity (FIG. 10C) waveforms in a common carotid artery, according to some embodiments;

FIGS. 11A-11C show example data of flow (FIG. 11A), diameter (FIG. 11B), and mean velocity (FIG. 11C) waveforms in an internal carotid artery, according to some embodiments;

FIG. 12A shows an electrical analogue of an example of a cerebral hemodynamics model with nonlinear, pressure-dependent cranial compliance, according to some embodiments, with the model shown for one data window where temporal variation in the values of the capacitors and resistors has been neglected. pₐ: Arterial pressure; q⁻: Arterial blood flow; C₁, C_{2,n}: Vascular and brain, and dural compliance; R: Vascular flow resistance; pᵥ: Venous pressure; pᵢ: ICP (CSF pressure);

FIG. 12B shows a plot of a nonlinear pressure-volume relationship for the compliance C_{2,n}, showing that the relationship can be linearized around an operating point (black dot) and the resulting affine approximation has a y-intercept of p_{dc} and a slope of 1/C₂, according to some embodiments; and

FIG. 12C shows the resulting linearized model from FIG. 12B, where the linear capacitors present an effective compliance, Ce.

### DETAILED DESCRIPTION

Systems and methods related to determination of patient parameters such as intracranial pressure (ICP), intracranial compliance (ICC), and cerebrovascular resistance (CVR) are provided. In some instances, the parameters are determined based at least in part on measures of two or more parameters related to distension of a vessel and blood flow through the vessel (e.g., arterial blood pressure and cerebral blood flow). In some instances, the parameters are determined based at least in part on measurements performed using one or more probes (e.g., a single probe) at a single patient site.

In some aspects, noninvasive systems and methods for determining intracranial pressure (ICP), intracranial compliance (ICC), and cerebrovascular resistance (CVR) measurement that can be deployed at the point-of-care (POC) are provided. The system may be configured to take measures such as invasive or noninvasive arterial blood pressure (ABP) and volumetric cerebral blood flow (CBF) as input, e.g., with both measurements made simultaneously at a cerebral blood vessel or one feeding the cerebral vasculature, such as the internal carotid artery (ICA). These input measurements may be fed to a physiologically-based model of the relevant cerebrospinal/cerebrovascular relationships to obtain robust ICP, ICC, and CVR estimates.

Brain tissue lacks internal energy deposits and is highly vulnerable to reduced supply of metabolic substrates. A few seconds of oxygen deficit, for example, may trigger neurological symptoms with sustained oxygen deprivation over a few minutes resulting in severe and often irreversible brain damage. Such rapid dynamics coupled with the potential for severe injury necessitates continuous, and ideally noninvasive, cerebrovascular monitoring in patients with or at greatest risk of developing brain injury. Intracranial pressure (ICP) is the hydrostatic pressure of the cerebrospinal fluid and is a key variable of interest in patients with head injuries as increased ICP causes brain tissue distortion and also impedes cerebral blood flow. ICP elevations can occur in a variety of neuropathological conditions, such as hydrocephalus, traumatic brain injury, hemorrhagic stroke, and brain tumor formation for example. The intracranial compliance (ICC) is another variable of interest and represents the propensity of rise in ICP in response to shifts in intracranial compartmental volumes. ICC has potential, therefore, to serve as an early warning for impending ICP elevation. If the ICC is high, intracranial space filling lesions can be buffered and result in comparatively little change in ICP. If ICC is low, however, any incremental volume increase in a space-filling lesion results in a dramatic increase in ICP. The resistance presented by cerebral vessels to blood flow, known as the cerebrovascular resistance (CVR), is also important in diagnosing vascular disorders and the cerebrovascular system's response to injury (e.g., spasms of cerebral arteries).

The current clinical gold-standard of ICP measurement involves drilling a hole through the skull to place a pressure sensitive probe or a fluid-filled catheter in the brain parenchyma or cerebral fluid spaces. Additionally injecting a known volume of fluid into the cerebral space and observing the rise in ICP leads to an assessment of the ICC. ICP measurements must be combined with those of the ABP and CBF to estimate the CVR. The invasive and infection-prone nature of such measurements means that only patients for whom the benefits of knowing ICP, ICC and CVR outweigh the risks associated with such invasive measurements undergo ICP/ICC/CVR monitoring. These are typically patients with potentially life-threatening cerebrovascular/cerebrospinal conditions, despite evidence that a larger pool of patients may benefit from such assessment. This insight has prompted the development of noninvasive alternatives. Therefore, systems and methods that can facilitate a noninvasive measurement of ICP, ICC, and/or CVR (e.g., using a probe at a patient site) and communicate such a measurement in a manner that effectively guides clinical care represent a marked technological improvement. Aspects of the present disclosure describe implementations that can, in some instances, achieve this improvement.

Examples of noninvasive ICP estimation methods include the application of external pressure on the eyeball to balance tissue pressure with ICP, using transcranial Doppler (TCD) ultrasound-derived indices, and statistics/machine-learning based methods. Transcranial acoustic signal properties have also been investigated for ICP estimation. Reliable indices of the ICC are currently lacking, prompting use of surrogates such as ICP waveform pulsatility.

One non-invasive technique uses physiologic model-based methods that relate a subjects' ABP with cerebral blood flow velocity (CBFV) and ICP. The ABP is measured at a peripheral location such as the fingers or the wrist using noninvasive means (finger-cuff inflation) or using minimally invasive indwelling catheters. The CBFV is measured using TCD in a cerebral artery. In this technique, a patient-specific three parameter model of cerebral blood flow is applied, where the three model parameters are relative resistance to blood flow (R), relative compliance (lack of stiffness) of the blood vessels and brain tissue (C), and the mean absolute ICP (l). It has been shown that these three parameters can be estimated in real-time using the ABP and CBFV signals without the need for prior training. The training-free nature, combined with the ease of interpretation of the model parameters make this technique potentially attractive for noninvasive ICP estimation. This technique can also incorporate its model-based approach within a probabilistic framework for increased robustness whilst retaining the interpretability and computational simplicity of our previous methods. The resulting scheme has achieved a mean error or accuracy of 0.6 mmHg and a root-mean-square error of 3.7 mmHg on data from thirteen patients at Boston Children's Hospital. When pooling data from three Boston-area hospitals, the model-based approaches can achieve an accuracy of 0.5 mmHg and a root-mean-square error of 5.0 mmHg in 29 subjects, ranging in age from 2 to 85 years and representing a diverse set of primary indications for invasive ICP monitoring (hydrocephalus, hemorrhagic stroke, severe traumatic brain injury, cerebrovascular malformations, brain tumor, and metabolic disorders).

Model-based approaches may be mechanistic and be capable of estimating model parameters without requiring a set of training or population level data. Numerous examples of model-based approaches are described in detail below.

It has been realized in the context of this disclosure that, in at least some instances, measures of two or more parameters related to distension of a vessel and blood flow through the vessel can be acquired (e.g., jointly acquired) at a single measurement site (a patient site). For example, it has been realized in the context of this disclosure that, in at least some instances, both ABP and CBF waveforms can be jointly acquired at a single measurement site (a patient site), e.g., using a standard clinical-grade ultrasound imaging probe. Doing so may allow one to conveniently deploy such a device at the POC. Therefore, systems configured to obtain a set of data, based at least in part on information about measurements at a single patient site (e.g., using one or more probes such as a single probe) and identify measures of two or more such parameters (e.g., ABP and volumetric CBF) may facilitate the use of more convenient and/or efficient devices to measure ICP, and/or CVR and, in some instances prompt and/or guide treatment of subjects (e.g., at a point of care). This is in contrast to techniques where a separate measurement device is used for ABP measurement at a peripheral location (wrist or finger) rendering the method potentially less amenable for POC monitoring.

Some embodiments involve use of CBF instead of CBFV. It has been realized in the context of this disclosure that doing so can, at least in some instances, allow one to noninvasively estimate the ICC and the CVR in absolute units of mL/mmHg and mmHg·s/mL, respectively. Absolute measurements may allow for long-term inter- and intra-subject comparisons that are not possible with relative measurements.

Some techniques allow determination of mean ICP only. However, some embodiments implementing the teachings of this disclosure can facilitate a non-invasive model-based method for the ICP waveform estimation.

Aspects of the disclosure relate to special purpose computers programmed to perform the algorithms described herein. For example, a special purpose computer may be programmed with instructions for carrying out the algorithms described herein for determining at least one of ICP, ICC, or CVR, among other algorithms provided herein. In some embodiments, a special purpose computer comprises a system including at least one hardware processor and at least one non-transitory computer-readable storage medium, among other components.

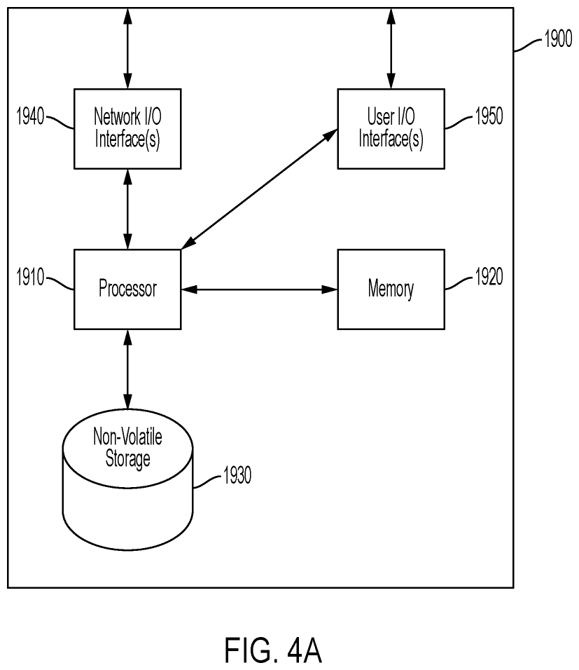

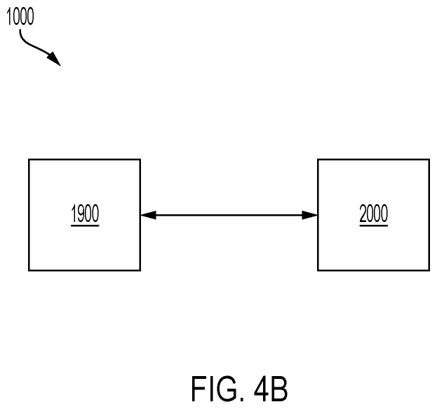

Some aspects of this disclosure relate to systems comprising at least one hardware processor. Some aspects relate to non-transitory computer-readable storage media storing processor-executable instructions that, when executed by the at least one hardware processor, cause the at least one hardware processor to perform steps relating to obtaining one or more sets of data and outputting information. FIGS. 4A-4B show illustrative block diagrams of some embodiments of such systems, which are described in more detail below. The system may use the set of data for identifying measures of two or more parameters related to distension of a vessel and blood flow through the vessel. Non-limiting parameters that can satisfy these criteria include, but are not limited to a measure of arterial blood pressure and a measure of cerebral blood flow of a patient. As noted above, the measure of the cerebral blood flow may be cerebral blood flow velocity (CBFV). Alternatively or additionally, in some embodiments, the measure of the cerebral blood flow is or includes volumetric cerebral blood flow (CBF). CBF may provide certain advantages in some instances. For example, the system may identify a measure of CBF (e.g., as a waveform) as opposed to CBFV, which can allow for subsequent or simultaneous calculation of ICC and/or CVR (e.g., in absolute units), which, it has been realized in the context of this disclosure, can be advantageous compared to the use of relative measurements. Non-limiting techniques for making measurements and calculating parameters relating to distension of a vessel and/or blood flow through the vessel are described in more detail below.

In some embodiments, the measurements determined at the patient site used to create information that can be sent to the hardware processor include a B-mode measurement (e.g., at a cerebral vessel). In some embodiments, the measurements determined at the patient site used to create information that can be sent to the hardware processor include a color flow measurement (e.g., at a cerebral vessel). These measurements may be used or determined using at least one sensor. For example, these measurements may be used with a probe such as an ultrasound probe. In some, but not all embodiments, at least some of the measurements are calibrated based on data from, for example, an arm-cuff. In other embodiments, other sensors may be used to determine such measurements.

In some embodiments, development of a single-probe ultrasound-based device for ICP, ICC, and CVR measurement at the POC is described. Ultrasound images may be acquired using standard clinical-grade imaging devices and used to determine both vessel diameter and CBF waveforms. The diameters may be transformed to ABP waveforms using calibration to intermittent arm-cuff-based ABP measurements or through pre-trained or first-principle models. The resulting ABP and CBF waveform pairs may then be used to determine the ICP waveform along with estimates for the ICC and CVR in a model-based fashion. It is believed that this technology constitutes a step forward towards reliable ubiquitous noninvasive neuromonitoring.

As mentioned elsewhere in this disclosure, the data obtained from the measurements received by the hardware processor may identify a measure of arterial blood pressure. For example, this data may be processed according to algorithms described herein to output a measure of arterial blood pressure. In some such embodiments, the data comprise arterial blood pressure waveforms. The arterial blood pressure waveforms may be converted from vessel diameter waveforms. Such a conversion may be performed in any of a variety of manners by the hardware processor. For example, the instructions, when executed, may cause the hardware processor to use a pre-trained model to perform the conversion of a vessel diameter waveform to an arterial blood pressure waveform. As another example, the instructions, when executed, may cause the hardware processor to use a first-principle model to perform the conversion of a vessel diameter waveform to an arterial blood pressure waveform. As yet another example, the instructions, when executed, may cause the hardware processor to use a calibration with an arm-cuff to perform the conversion of a vessel diameter waveform to an arterial blood pressure waveform. In some instances, a combination of these techniques are used. Non-limiting examples of model-based techniques for such a conversion are described below.

As also mentioned elsewhere in this disclosure, the data obtained from the measurements received by the hardware processor may be processed to identify a measure of cerebral blood flow (e.g., volumetric cerebral blood flow). In some such embodiments, the data are calculated via integration of a measure of cerebral blood flow velocity (CBFV). The integration may be performed, for example, using vessel diameter waveforms. Non-limiting examples of model-based techniques for such an integration are described below.

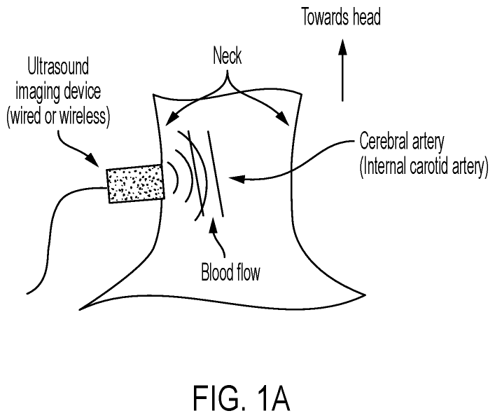

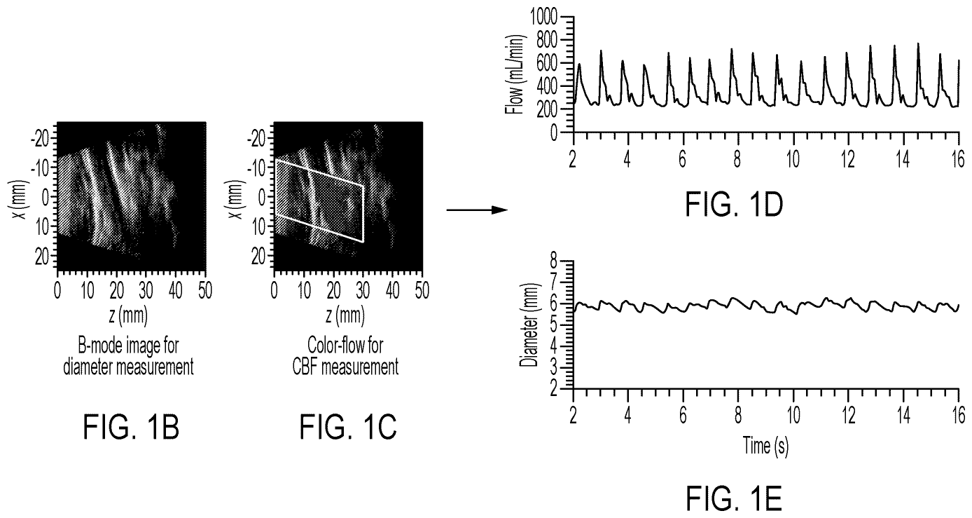

FIGS. 1A-1E illustrate a system according to some embodiments. In this example system, a clinical-grade ultrasound imaging device is used to perform standard (time-interleaved) B-mode and color flow measurements at a cerebral vessel (for instance, the ICA). The B-mode images are processed to detect vessel edges and estimate vessel diameters. The velocity measurements from the color flow data are then integrated to estimate the volumetric flow. Vessel diameters have been shown to correlate with ABP waveforms. Thus, the diameter waveforms are converted to ABP waveforms using pre-trained models, first-principle models, or by calibrating with an armcuff. This calibration may be performed intermittently (for instance, once every five minutes). ABP and CBF waveforms in 15- to 60-second recording windows are then passed to a model-based estimation scheme as described in S. M. Imaduddin, Ultrasound-based noninvasive monitoring methods for neurocritical care, Massachusetts Institute of Technology, 2022, which is incorporated by reference in its entirety for all purposes.

One non-limiting example of a model-based estimation scheme that uses color flow measurements and B-mode images at a vessel is described as follows.

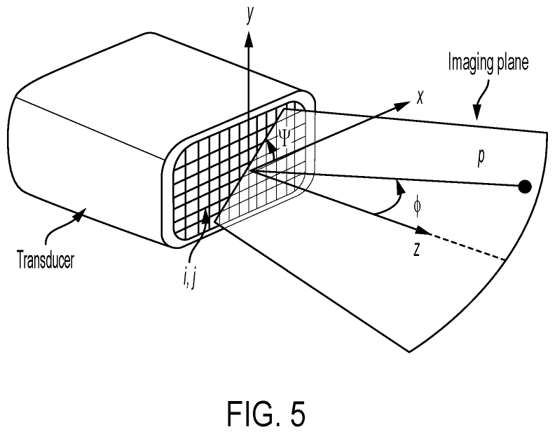

Interleaved B-mode and color flow (CF) frames can be acquired by transmitting ultrasound beams over a range of directions in a 2D plane with rotation angle ψ=0° (FIG. 5). Received, demodulated echoes for the k^{th} B-mode and CF frame pair can be denoted as

$${r}_{p , φ}^{k} and {\check{s}}_{p , φ}^{k} [ n ] ,$$

respectively, where n is the ensemble index for CF data, and ρ is the radial distance from the origin along the azimuth angle, φ. A detrending procedure may be used to remove clutter from the CF data, and clutter-filtered CF data may be denoted

$${s}_{p , φ}^{k} [ n ] .$$

Non-angle-corrected CF velocity estimates,

$${\check{v}}_{p , φ}^{k} ,$$

for an ensemble, E, of focused transmitted beams can be computed using an autocorrelation-based estimator in Equation 1.

$$v = \frac{cPRF}{4 π {f}_{0}} arg \{ {Γ}_{s s} ( 1 ) \} \qquad ( 1 )$$

where Γₛₛ(1) is an estimate of the unit-lag autocorrelation of s[n] such that

$${Γ}_{s s} ( 1 ) = \frac{1}{E - 1} {\sum }_{n = 1}^{E - 1} {s}^{*} [ n ] s [ n + 1 ] \qquad ( 2 )$$

where (*) denotes complex conjugation. In Equation 1, “PRF” refers to the pulse repetition frequency. In some embodiments, the pulse repetition frequency is 50000 pulses per second. In Equation 1, “c” refers to the speed of sound in the medium. For example, the speed of sound in human tissue is typically 1500 m/s. In Equation 1, “f₀” refers to the insonation center frequency. In some embodiments, f₀ is greater than or equal to 4 MHz and less than or equal to 6 MHz for soft-tissue ultrasound.

The corresponding signal power can be computed using Equation 3 and denoted

$${P}_{p , φ}^{k} .$$

$$P = \frac{1}{E} {\sum }_{n = 1}^{E} {\vert s [ n ] \vert}^{2} \qquad ( 3 )$$

Flow unidirectionality,

$${U}_{p , φ}^{k} ,$$

can be computed according to

$$U = max ( \frac{{P}_{\geq 0}}{{P}_{< 0}} , \frac{{P}_{< 0}}{{P}_{\geq 0}} ) \qquad ( 4 )$$

where the power in the positive and negative frequency bands is denoted P_{≥0} and P_{<0}, respectively, and dependence on k, ρ, and φ is omitted for clarity. P_{≥0} and P_{<0} may be determined by summing the positive and negative frequency bins of power spectral densities of

$${s}_{p , φ}^{k} [ n ]$$

computed using the Welch method as described in A. V. Oppenheim and R. W. Schafer. Discrete-Time Signal Processing, 3rd edition. Prentice Hall, 2002, which is incorporated herein by reference.

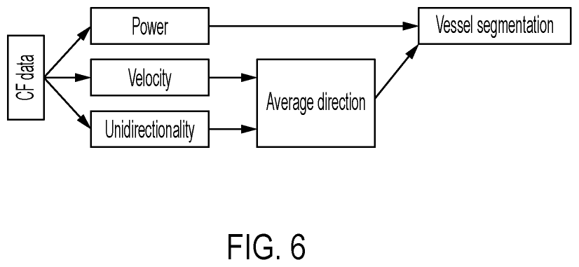

CF-derived velocity, power, and unidirectionality may be used to coarsely segment the target vessel. B-mode images may then be processed to extract the vessel location, diameter, and orientation with greater precision. The velocity signatures within the detected vessel lumen may be angle-corrected and integrated to yield the volumetric flow. The point spread function may be ignored when higher frequency ultrasound waves (>4 MHz) are employed, such as with target vessel diameters exceeding 3 mm. CF data may first be used to coarsely localize the target vessel (FIG. 6). Power, velocity, and unidirectionality in each CF frame may first be mapped to Cartesian coordinates, x, and z. Carotid CF scans often contain adjacent signatures from arteries and veins. Moreover, arterial systolic velocity can occasionally exceed the maximum detectable velocity set by the PRF of the system, appearing as a negative velocity instead. It is therefore helpful in some instances to separate venous flow from arterial flow, while ignoring the occasional appearance of negative velocity within the arterial lumen. A direction mask,

$${\overline{D}}_{x , z}^{k} ,$$

representing the average direction of flow, may therefore be used.

First, the instantaneous flow direction,

$${D}_{x , z}^{k} ,$$

can be determined according to

$${D}_{x , z}^{k} \{ \begin{matrix}+ {U}_{x , z}^{k} , & {\check{v}}_{x , z}^{k} \geq 0 \\ - {U}_{x , z}^{k} , & {\check{v}}_{x , z}^{k} < 0\end{matrix} \qquad ( 5 )$$

The average direction of flow,

$${\overline{D}}_{x , z}^{k} ,$$

may then be determined using a single-tap IIR filter of the form

$${\overline{D}}_{x , z}^{k} = γ {\overline{D}}_{x , z}^{k} + ( 1 - γ ) {D}_{x , z}^{k} \qquad ( 6 )$$

where γ is a filtering coefficient, set to 0.9. The set of pixels corresponding to the arterial lumen can be denoted C^{k} such that

$${C}^{k} = \{ ( x , z ) \vert {P}_{x , z}^{k} > {P}_{min}^{k} , {\overline{D}}_{x , z}^{k} \geq {D}_{min} \} \qquad ( 7 )$$

Where Dₘᵢₙ=2, and

$${P}_{min}^{k}$$

may be set to the 25^{th} percentile of the CF power in the selected subset. The largest connected component,

$${C}_{1}^{k} ,$$

within C^{k} may be retained for subsequent processing. For each unique x₁ in

$${C}_{1}^{k} ,$$

the minimal and maximal z-coordinates in

$${C}_{1}^{k}$$

may be selected and corresponded to coarsely segmented proximal and distal edge locations, respectively. The z-coordinates may be denoted

$${z}_{C F}^{p} ( {x}_{1} ) and {z}_{C F}^{d} ( {x}_{1} ) ,$$

respectively. It should be noted here that this approach cannot distinguish signatures from individual arteries, and manual intervention may be helpful to select the desired vessel if multiple arteries are present in the same field of view.

B-mode data may first be converted to Cartesian coordinates and denoted

$${r}_{x , z}^{k} .$$

Pixels inside a vessel lumen appear as darker regions in B-mode images. Thus, a data-derived blood-pixel threshold, B^{k}, may first be derived

$${B}^{k} = min ( {B}_{max} , {B}_{90}^{k} ) \qquad ( 8 )$$

where Bₘₐₓ=25 dB and

$${B}_{90}^{k} = {percentile}_{90} ( 20 {log}_{10} \vert {r}_{x 1 , z 1}^{k} \vert ) , ( {x}_{1} , {z}_{1} ) ∈ {C}_{1}^{k} \qquad ( 9 )$$

Pixels in the B-mode frame whose corresponding log-transformed magnitude is above

$${B}_{90}^{k}$$

may be denoted B^{k} where

$${B}^{k} = \{ ( x , z ) \vert 20 {log}_{10} \vert {r}_{x , z}^{k} \vert > {B}^{k} \} \qquad ( 10 )$$

Pixels in B^{k} with x-coordinate x₁, and z-coordinate closest to

$${z}_{C F}^{p} ( {x}_{1} ) and {z}_{C F}^{d} ( {x}_{1} )$$

may then be selected and denoted

$${z}_{B}^{p} ( {x}_{1} ) and {z}_{B}^{d} ( {x}_{1} ) ,$$

respectively. The sets of proximal, ε^{p}, and distal, ε^{d}, edges of the vessel may then be determined

$${ε}^{p} = \{ ( {x}_{1} , {z}_{B}^{p} ( {x}_{1} ) ) \vert - {z}_{max} < {z}_{B}^{p} ( {x}_{1} ) - {z}_{C F}^{p} ( {x}_{1} ) < {z}_{max} \} \qquad ( 11 )$$

$${ε}^{d} = \{ ( {x}_{1} , {z}_{B}^{d} ( {x}_{1} ) ) \vert - {z}_{max} < {z}_{B}^{d} ( {x}_{1} ) - {z}_{C F}^{d} ( {x}_{1} ) < {z}_{max} \} \qquad ( 12 )$$

where zₘₐₓ can be set to exclude points where the B-mode-based z-coordinates differ from the corresponding CF-based values. Vessel edges may be modeled as straight-line segments, and the random sample consensus (RANSAC) algorithm may be used to remove remaining outliers. A set of vessel centers, V, may then be determined using the resulting edges

$$𝒱 = \{ ( x , \frac{{z}^{p} + {z}^{d}}{2} ) ( x , {z}^{p} ) ∈ {ℰ}^{p} , ( x , {z}^{d} ) ∈ {ℰ}^{d} \} \qquad ( 13 )$$

A straight line may be fitted to coordinates in V in a least-square sense and the slope of the line may be used to estimate the angle, θ^{k}, between the CF-beams and the vessel. The spatial-average vessel diameter, d[k], can also be determined.

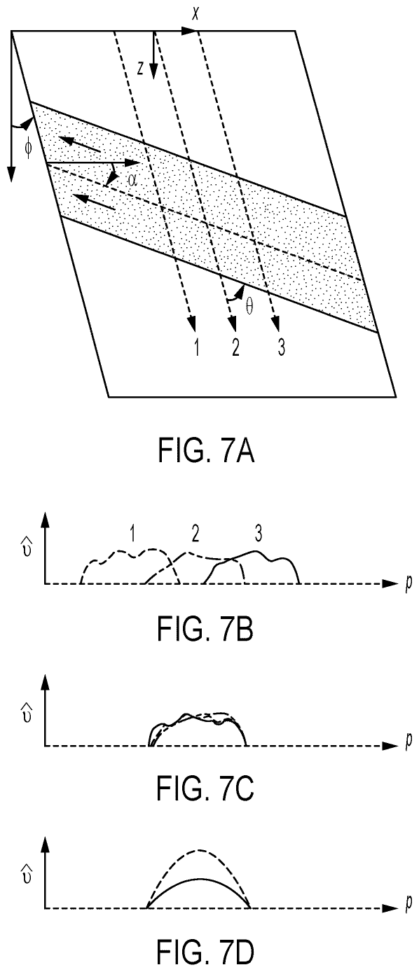

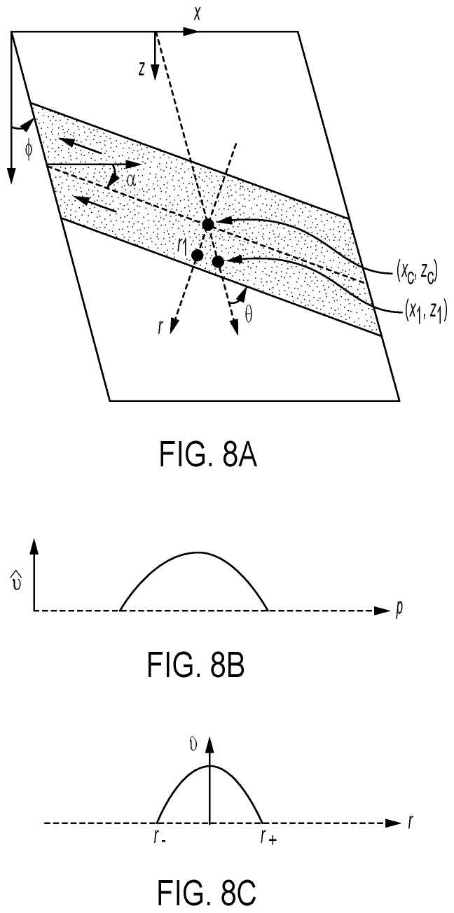

CF data may be processed to extract an average velocity profile in the segmented vessel lumen. Negative velocities may first be wrapped around to positive values as necessary. Velocity profiles measured along each received direction may be spatially aligned and averaged as shown in FIGS. 7A-7D. The averaged velocity profile may have erroneous values at the edges due to low CF power. Thus, a smoothed velocity profile may be determined by fitting a spline in a least-square sense and scaled by cos⁻¹(θ^{k}). The velocity profile may be projected onto the vessel's radial axis (FIGS. 8A-8C) and the resulting scaled velocity profile, \hat{v}^{k}(r), may then be integrated to yield the instantaneous volumetric flow

$${\dot{q}}_{inst} [ k ] = π {\int }_{{r}_{-}}^{{r}_{+}} {\hat{v}}^{k} ( r ) rdr \qquad ( 14 )$$

where r₋ and r₊ are the vessel extents along the radial coordinate, r, from the center of vessel, \hat{v}^{k}(r) is the corresponding angle-corrected velocity, and the vessel is assumed to be circular. A spatial-average velocity may also be computed as

$$\overline{v} [ k ] = \frac{{\dot{q}}_{inst} [ k ]}{π {d [ k ]}^{2} / 4} \qquad ( 15 )$$

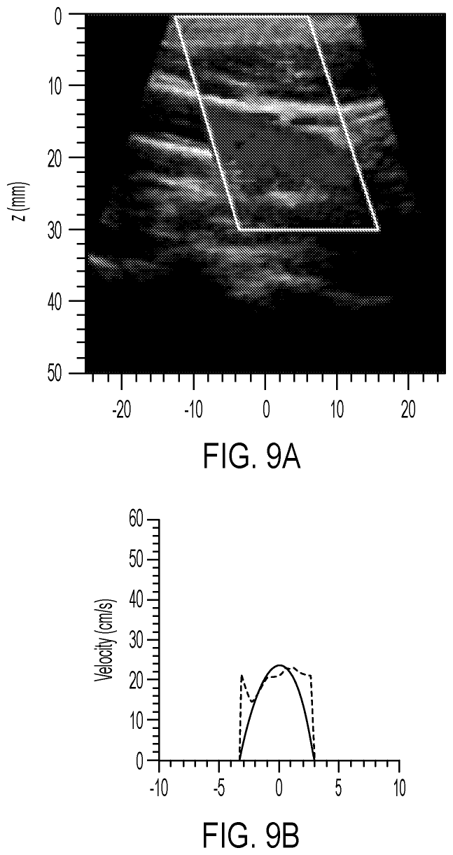

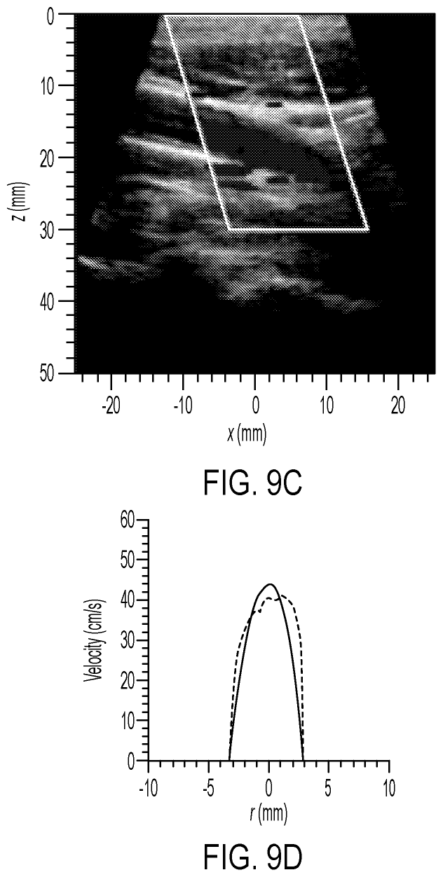

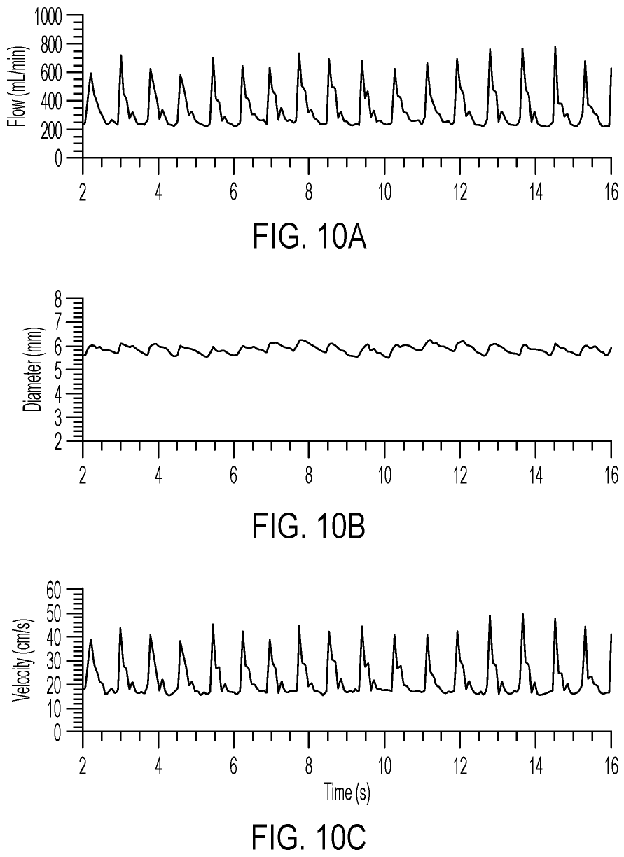

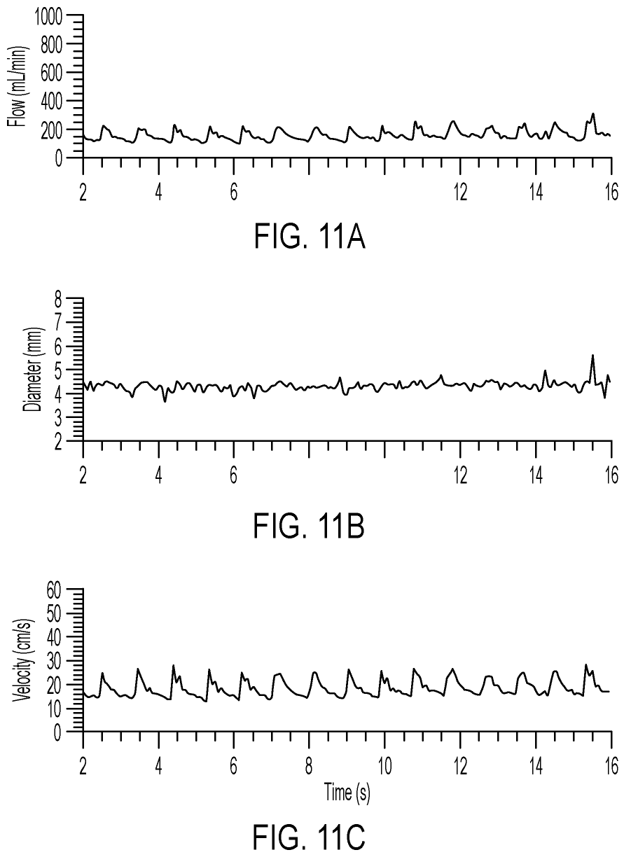

FIGS. 9A-9D show examples of the velocity profiles in two frames, while FIGS. 10A-10C and FIGS. 11A-11C show examples of waveforms recorded from a subject's common and internal carotid artery (ICA), respectively. In vessels like the ICA, the diameter measurements often contain artifacts as the vessel edges may not be clearly detected. It was observed that while the resulting volumetric flow waveforms were also corrupted with the same artifacts, the spatial-average velocity waveforms largely remained artifact-free (as may be seen in FIGS. 11A-11C). Thus, an alternate volumetric flow waveform was also computed by multiplying the spatial-average velocity with the time-averaged vessel cross-section area.

$$\dot{q} [ k ] = \frac{π}{4} \overline{v} [ k ] {\overline{d}}^{2} \qquad ( 16 )$$

where d̅ is the time-averaged vessel diameter. The resulting volumetric flow waveform, \dot{q}[k], contains the expected pulsatile flow at the expense of inaccuracy in the instantaneous measurements.

The imaging procedure described above may, in some instance, be repeated for at least one other rotation angle (ψ) for appropriate placement of the ultrasound probe. For example, a biplanar imaging sequence may be used. In some embodiments, a biplanar imaging sequence is used where ultrasound images are recorded for ψ=0° and 90°. Some such embodiments may permit the operator to place the ultrasound probe such that the vessel diameter and its pulsation are measured robustly along with the resulting volumetric flow.

As noted elsewhere in this disclosure, some embodiments involve outputting information indicating a measure of a value of intracranial pressure (ICP) (e.g., based at least in part on the set of data obtained by the processor (e.g., from measurements from one or more probes)). ICP refers to the hydrostatic pressure of cerebrospinal fluid (CSF), which is the fluid that surrounds and cushions the brain tissue of a human or animal. When the ICP becomes elevated in an individual, blood flow to the brain can become limited and lead to cerebral ischemic injury. Additionally, brain structures may become displaced (herniation) because of pressure differences within the cranial cavity and spinal canal, which may potentially lead to coma, cessation of breathing, and/or death. Elevation of ICP may occur in various neuropathological conditions, including hydrocephalus, traumatic brain injury, hemorrhagic stroke, brain tumors, and/or metabolic conditions. In managing these types of neuropathological conditions, it can be important to monitor the ICP of the individual to assess the cerebrovascular and cerebrospinal state of the individual and to determine if the ICP becomes elevated to a point that puts the individual at a high-risk level.

The measure of the value of ICP output by the hardware processor in certain systems of this disclosure may be in absolute units and/or in relative units. In the context of this disclosure, absolute units are understood to refer to the absolute physiologically relevant units corresponding to that parameter, as opposed to non-physiologically relevant units that merely correlate with the value of that parameter. For example, absolute units of ICP refer to units of pressure, which are expressed in terms of force divided by area. Accordingly, in some embodiments, the measure of a value in absolute units of ICP is in the units of mmHg, pascals, bars, pounds per square inch (PSI), atmospheres, or a multiple thereof. An example of a multiple of the aforementioned units is any unit that can be obtained by simply multiplying the units by a number. For example, a kilopascal is a multiple of pascal because it is obtained by multiplying a value in units of pascals by 0.001. An example of a measure of ICP that is not in absolute units in the manner with which the term is used in the context of this disclosure would be an ultrasound time-of-flight measurement through a subject's skull. The time-of-flight measurement is expected to be correlated with ICP to the extent that a faster time-of-flight is typically associated with a higher ICP. However, a measure of time-of-flight (in units of time), while correlated with ICP and therefore a relative measure of ICP, is not in physiologically relevant units of pressure and would not generally be useful in guiding monitoring and/or treatment of a patient. By contrast, absolute units in terms of pressure (force divided by area) would be useful for guiding monitoring and/or treatment of a patient.

As discussed elsewhere in this disclosure, obtaining and/or outputting (e.g., communicating) values of parameters such as ICP, ICC, and/or CVR in absolute units can be advantageous because it can promote long-term inter- and intra-subject comparisons that are not possible with relative measurements. Furthermore, output of ICP, ICC, and/or CVR in absolute units can improve care of patients suffering from or suspected of suffering from a neuropathological condition at least because clinical guidelines for monitoring and/or treatment tend to be conveyed in terms of absolute units, and so a system that output such information in absolute units may be more clinically actionable than those that output relative units.

As noted elsewhere in this disclosure, some embodiments involve outputting information indicating a measure of a value of intracranial compliance (ICC) (e.g., based at least in part on the set of data obtained by the processor (e.g., from measurements from one or more probes)). Intracranial compliance (ICC) is a parameter refers to the rise in intracranial volume in response to an increase in ICP. ICC therefore captures the buffering capability of the system. A comparatively high ICC value suggests that buffering may still be adequate to compensate for intracranial volume shifts (e.g., maintaining ICP relatively constant). Conversely, a comparatively low ICC value can indicate that, for the same volume shift, ICP will increase more significantly. Accordingly, ICC can help characterize a subject's operating point on the “pressure-volume” curve, and can in some instances offer a potential early warning of rising ICP. Accordingly, in some instances, outputting of a measure of ICP and outputting a measure of ICC can, in combination, provide greater insight into treatment choice for a subject than ICP alone. A reliable and convenient system to measure and/or monitor ICC in addition to ICP (e.g., at a patient's bedside) could be helpful in determining whether the brain is in a compensated (stable or low-risk) state or in an uncompensated (unstable or high-risk) state in relation to further incremental volume increases. Determining ICC according to the embodiments described in the disclosure (e.g., based on information from measurements of the distension of a vessel and/or blood flow through the vessel) may be less invasive and therefore more convenient and accessible than convention ICC measurement techniques such as volume-pressure tests in which fluid boluses are injected into the craniospinal space and ICP is concomitantly measured.

The measure of the value of ICC output by the hardware processor in certain systems of this disclosure may be in absolute units and/or in relative units. Absolute units of ICP refer to units of volume divided by pressure, which are expressed in terms of volume divided by a quantity of force divided by area. Accordingly, in some embodiments, the measure of a value in absolute units of ICC is in the units of mL/mmHg, mL/pascal, mL/bar, mL/PSI, mL/atmosphere, or a multiple thereof. In some embodiments, the measure of the value of ICC in absolute units is a measure of a value in absolute units of a regional ICC. However, the measure of the value of ICC in absolute units is a measure of a value in absolute units of a global ICC.

As noted elsewhere in this disclosure, some embodiments involve outputting information indicating a measure of a value of cerebrovascular resistance (CVR) (e.g., based at least in part on the set of data obtained by the processor (e.g., from measurements from one or more probes)). CVR refers to the cerebral perfusion pressure (which is the difference between arterial blood pressure and downstream pressure) divided by volumetric cerebral blood flow (CBF).

The measure of the value of CVR output by the hardware processor in certain systems of this disclosure may be in absolute units and/or in relative units. Absolute units of CVR refer to units corresponding to pressure divided by a quantity of volume divided by time for CVR. Accordingly, in some embodiments, the measure of a value in absolute units of ICC is in the units of mmHg/(mL/min.), mmHg/(mL/s), pascal/(mL/min.), PSI/(mL/min.), atmosphere/(mL/min.), or a multiple thereof. In some embodiments, the measure of the value of CVR in absolute units is a measure of a value in absolute units of a regional CVR. However, the measure of the value of CVR in absolute units is a measure of a value in absolute units of a global CVR.

As discussed elsewhere in this disclosure, some embodiments are directed to systems configured to acquire measurements at a patient site—that is a single patient site as opposed to measurements from multiple different patient sites—that can be used (e.g., via the hardware processor) to obtain a set of data indicative of two or more parameters (e.g., of distension of a vessel and/or blood flow through the vessel). Contrary to existing configurations for obtaining measurements for calculating such parameters based on measurements from different sites, it has been realized in the context of the present disclosure that sufficient measurements can be obtained at a single patient site. This can, in some instances, allow for a reduction in complexity of the measurements and number of devices needed, which can improve ease and convenience. In some such instances, the ability to obtain measurements at a single patient site facilitates the system being a point of care system (e.g., a non-invasive, real-time, patient-specific system for measuring intracranial pressure (ICP), intracranial compliance (ICC), and/or cerebrovascular resistance (CVR)).

As one example of a possible advantage of performing measurements at a single patient site, information about the measurements may be sent to the hardware processor, which may be configured to obtain a set of data identifying a measure of arterial blood pressure and a measure of cerebral blood flow (e.g., volumetric cerebral blood flow) of a patient based on that information. Those measures of arterial blood pressure and cerebral blood flow may then be used to calculate and output measures indicative of values of ICP, ICC, and/or CVR. By contrast, existing systems configured to perform measurements of arterial blood pressure and cerebral blood flow do so via separate measurements at different patient sites—a first measurement at a first patient site to determine ABP (e.g., using a radial arterial catheter at the wrist) and a second measurement at a second patient site to determine cerebral blood flow velocity (e.g., using a transcranial Doppler (TCD) at the middle cerebral artery (MCA)). Accordingly, single patient site measurements as described in this disclosure may advantageously promote more facile measurements. Additionally, the performance of measurements at a single patient site may reduce or eliminate a need for temporal alignment of measurements when calculating the relevant parameters.

The patient site at which the measurements are performed may correspond to a single vessel or a portion thereof. The vessel may be an extracranial vessel or an intracranial vessel. Extracranial vessels may be particularly convenient for some embodiments. Examples of suitable vessels at which the measurements may be performed (e.g., in a localized manner) include, but are not limited to, a common carotid artery, an internal carotid artery, a vertebral artery, a basilar artery, a middle cerebral artery, a posterior cerebral artery, and/or an anterior cerebral artery.

The patient site at which the measurements are performed may correspond to a region of the patient having a relatively low volume. For example, the patient site may correspond to a spatial region of the patient (e.g., a spatial region of interrogation by the probe such as a spatial region of insonation) having a volume that is less than or equal to 5,000 mm³, less than or equal to 2,000 mm³, less than or equal to 1,000 mm³, less than or equal to 500 mm³, less than or equal to 200 mm³, less than or equal to 100 mm³, and/or as low as 50 mm³, as low as 10 mm³, or less. Combinations of these ranges are possible. In some embodiments, the patient site at which the measurements are performed corresponds to a region of the patient having a relatively low area (e.g., a low area subject to interrogation such as imaging). For example, the patient site may correspond to a region of the patient having an area of less than or equal to 10,000 mm², less than or equal to 5,000 mm², less than or equal to 2,500 mm², less than or equal to 1,000 mm², less than or equal to 750 mm², less than or equal to 600 mm², and/or as low as 500 mm², as low as 400 mm², as low as 300 mm², as low as 200 mm², as low as 100 mm², or lower. Combinations of these ranges are possible.

As mentioned above, the measurements that can be used to calculate the parameters related to distension of a vessel or blood flow through the vessel (e.g., ABP and CBF or CBFV) may be performed by one or more probes. As described below, a probe may comprise one or more sensors. The probe may be configured to perform the measurements at a patient site and send information about the measurements to the at least one hardware processor. Any of a variety of types of probes may be used. The probe may be a non-invasive probe. For example, the probe may not require the introduction of an instrument into the body of a subject. In some embodiments, the probe is an ultrasound probe (e.g., configured to perform ultrasound imaging). In some embodiments, the ultrasound probe is configured to perform color flow imaging measurements, B-mode measurements, and/or pulsed-wave spectral ultrasound measurements. One non-limiting example of an ultrasound probe that can be used in some embodiments is a Butterfly iQ handheld ultrasound device, details of which are described in Sanchez, N., et al., (2021 February). “34.1 an 8960-element ultrasound-on-chip for point-of-care ultrasound.” In 2021 IEEE International Solid-State Circuits Conference (ISSCC) (Vol. 64, pp. 480-482), which is incorporated herein by reference.

In some embodiments, the system comprises only a single probe configured to perform the measurements and send the information to the hardware processor. As discussed elsewhere in this disclosure, the use of a single probe configured to perform measurements at a single patient site to determine the parameters described in this disclosure may improve upon conventional technology by reducing the number of devices needed to make the measurements, which may afford greater convenience, affordability, and access to care (e.g., by allowing for a point-of-care system). In some embodiments, the single probe comprises multiple sensors (e.g., configured to perform different imaging such as B-mode imaging and color flow imaging). However, in some embodiments, the single probe comprises a single sensor. In some embodiments, the system comprises two or more probes configured to perform the measurements at the single patient site (e.g., two different ultrasound probes).

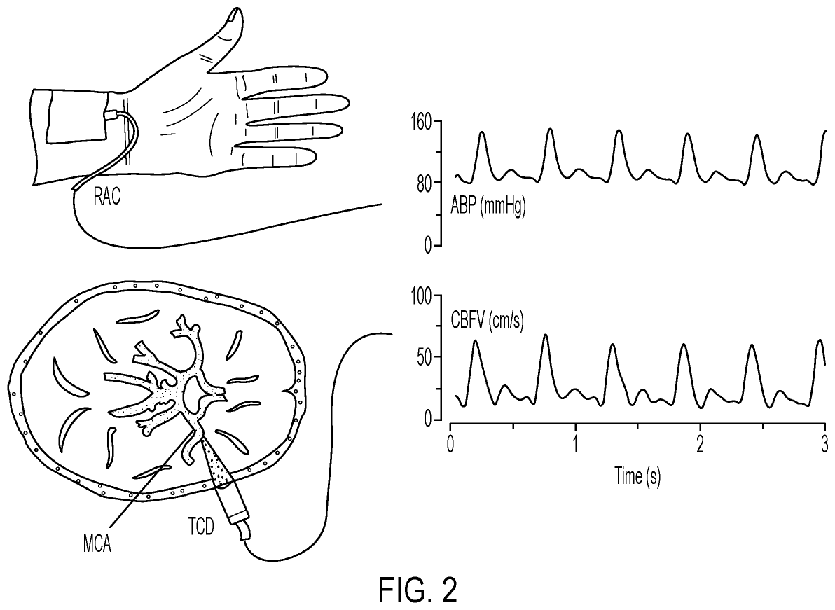

In some embodiments, a single probe (e.g., a single ultrasound imaging probe) is used and can therefore be performed easily at the POC. Other setups, such as that illustrated in FIG. 2 where two separate devices are used to measure ABP and CBFV at different anatomical locations may not be amenable for POC measurement. Measurements at two separate anatomical locations may also mean that the waveforms have to be temporally aligned prior to ICP estimation to account for both device-dependent and physiologic time offsets. By contrast, single-location measurement as used in some embodiments does not require such time-alignment, as measurements have a well-defined temporal relationship. Techniques such as those proposed in U.S. Patent Application Publication No. 20220160327 may therefore be rendered unnecessary. The method of U.S. Pat. No. 11,166,643 also advocates measuring flow in arteries outside the head but not at the same location. The ensuing ICP estimation technique also differs significantly.

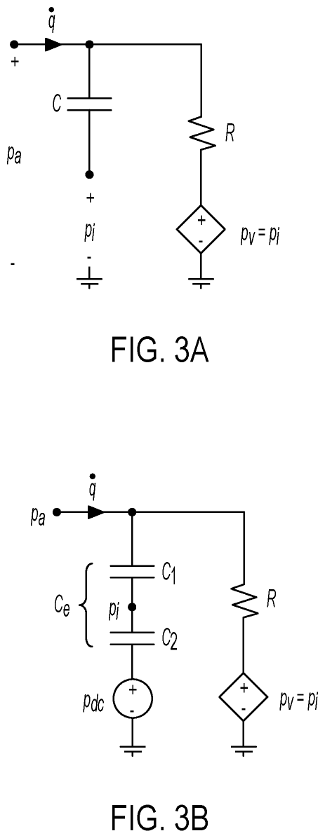

Another aspect of at least some embodiments of the present disclosure relates to determining the ICC and CVR in absolute units. While measurements in relative units may allow one to track relative changes in ICC and CVR within the same patient over time, having these measurements in absolute units may allow clinicians to compare a patient's ICC and CVR values against population norms and initiate treatment if the measured values start to deviate from such population norms. In some embodiments, obtaining absolute ICC and CVR values is facilitated by acquiring CBF waveforms instead of CBFV, where the CBF is here taken to be the product of the vessel area and the CBFV. In some embodiments, the values of ICP, ICC, and/or CVR are calculated by feeding arterial blood pressure and volumetric cerebral blood flow waveforms to a modified physiologic model. Any of a variety of modified physiologic models may be employed, such as a modified physiologic model of cerebral blood flow. For example, the measured ABP and CBF waveforms may be fed to a modified physiologic model of the cerebral blood flow shown in FIGS. 3A-3B. An appropriate parameter estimation technique can then be used to determine values for C₁, C₂, R, and the ICP, pᵢ. The values for C₂ and R are used to represent the ICC and CVR, respectively. Tilt-table studies were conducted in healthy volunteers, and it was found that these methods tracked changes in ICP, CVR, and ICC.

In some embodiments, one potential advantage of the modified physiologic model is that the ICP waveform can be determined. Specifically, the model may posit that the pulsatile ABP and ICP waveforms are related by the voltage divider formed by the two capacitors, C₁ and C₂. Once estimates have been found for the latter, the ICP waveform may be able to be estimated from the ABP waveform. An associated parameter estimation approach has been provided in S. M. Imaduddin, Ultrasound-based noninvasive monitoring methods for neurocritical care, Massachusetts Institute of Technology, 2022.

The following describes examples of non-limiting parameter estimation approaches that can be used in some embodiments. In the model shown in FIG. 3A, first used by Kashif, a capacitor C represents brain and vascular compliance seen from a cerebral vessel to the CSF space, while the flow resistance, R, represents the aggregate arterial (and proximal venous) resistance. A dependent voltage source models the Starling resistor phenomenon from the point of the cerebral vascular system where flow limitation occurs. This model was designed to allow robust online estimation of pᵢ using approximately 60 beats of measured waveform data. CSF production and absorption rates occur over a longer time scale and are therefore not modeled. Likewise, for each estimation window, the compliance and resistance are assumed to remain approximately constant. For a data window, the model may therefore be represented by a first-order constant coefficient differential equation of the form

$$\dot{q} ( t ) = \frac{1}{R} ( {p}_{a} ( t ) - {p}_{i} ( t ) ) + C \frac{d}{dt} ( {p}_{a} ( t ) - {p}_{i} ( t ) ) \qquad ( 17 )$$

Any of several estimation approaches may be used to determine values for pᵢ, R, C using this model. Jaishankar et al. used a three-element variant of this model that included an inductor to model fluid inertance in R. Jaishankar, A. Fanelli, A. Filippidis, T. Vu, J. Holsapple, and T. Heldt. “A spectral approach to model-based noninvasive intracranial pressure estimation.” IEEE J Biomed Health Inform, 24 (8): 2398-2406, 2020, which is incorporated herein by reference. In conventional, typical practice, CBF measurements are not available and the CBFV is used as a surrogate. Assuming that the CBFV and CBF are linearly related, the model can be expressed as

$$v ( t ) = \frac{1}{{R}^{′}} ( {p}_{a} ( t ) - {p}_{i} ( t ) ) + {C}^{′} \frac{d}{dt} ( {p}_{a} ( t ) - {p}_{i} ( t ) ) \qquad ( 18 )$$

where v(t), is the CBFV waveform, and R′ and C′ are pseudo-resistance and pseudocompliance values, respectively. The ICP can still be estimated since it is left unaffected in the equation, whereas only relative values for fluid flow resistance and compliance can be measured. In the original work by others, another simplifying assumption was invoked by ignoring pulsatility in the ICP waveform. Thus, the estimation routines estimated the mean ICP only. Jaishankar et al. attempted to determine ICP pulsatility as well as the mean using a pretrained mapping between ABP and ICP pulsatile waveforms. Likewise, it has been shown that a two-step process may also be used to coarsely determine the ICP pulsatility-estimates for the mean ICP, and R′ and C′ are first determined by ignoring ICP pulsatility before using the resulting transfer function between ABP, CBFV, and ICP to determine an approximate ICP waveform.

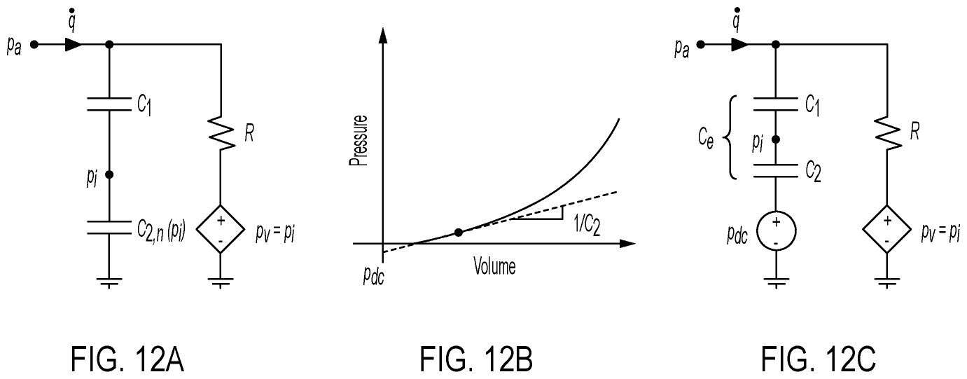

The Kashif model has been used to determine noninvasive ICP estimates in patients with several neurological conditions including traumatic brain injury. As mentioned above, however, the approach does not allow ICC determination since only the arterial (and brain) compliance is modeled, and the dural compliance is left unmodeled. Therefore, in some embodiments the original Kashif model is modified by including an additional nonlinear capacitor to represent dural compliance such that the voltage (pressure difference) across it becomes a nonlinear function of q₂, the charge (volume) stored in it (FIG. 12A). Linearizing the functional dependence between the volume and pressure difference around an operating point,

pᵢ≈p_{dc}+q₂/C₂ (19)

where p_{dc} is a fictitious DC pressure source, and C₂ is the incremental (linear) capacitance at the operating point (FIG. 12B). The resulting linearized circuit model is shown in (FIG. 12C, which matches FIG. 3B). For a data window, the circuit dynamics may be expressed as

$$q ( t ) = \frac{1}{R} ( {p}_{a} ( t ) - {p}_{i} ( t ) ) + C \frac{d}{dt} ( {p}_{a} ( t ) - {p}_{dc} ) = \frac{1}{R} ( {p}_{a} ( t ) - {p}_{i} ( t ) ) + C \frac{d}{dt} {p}_{a} ( t ) \qquad ( 20 )$$

where Cₑ is the ‘effective’ capacitance formed by the series interconnection of C₁ and C₂

$${C}_{e} = {( \frac{1}{{C}_{1}} + \frac{1}{{C}_{2}} )}^{- 1} \qquad ( 21 )$$

This model relates the pulsatile components of the ABP and ICP waveforms according to

$${\tilde{p}}_{i} ( t ) = \frac{{C}_{e}}{{C}_{2}} {\tilde{p}}_{a} ( t ) \qquad ( 22 )$$

where the tilde is used to denote mean-subtracted waveforms. Thus, values for C₁ and C₂ can be determined if Cₑ, \tilde{p}ᵢ(t), and \tilde{p}ₐ(t) can first be estimated.

The estimation approach described above uses the model in Equation 20 and can be adapted for the case when CBFV is used as a surrogate for CBF. For the m^{th} data window, a first-order finite-difference approximation for the derivative term can be used to obtain

$$q [ k - {k}_{o} ] = \frac{1}{{R}_{m}} ( {p}_{a} [ k ] - {\tilde{p}}_{i} [ k ] - {\overline{p}}_{i} [ m ] ) + {C}_{e , m} {f}_{s} ( {p}_{a} [ k ] - {p}_{a} [ k - 1 ] ) = {α}_{m} ( {p}_{a} [ k ] - {\tilde{p}}_{i} [ k ] - {\overline{p}}_{i} [ m ] ) + {β}_{m} ( {p}_{a} [ k ] - {p}_{a} [ k - 1 ] ) \qquad ( 23 )$$

where fₛ is the sampling rate, p̅ᵢ̅[m] is the mean ICP for the data window, \tilde{p}ᵢ[k] is the pulsatile component of the ICP, αₘ=1/Rₘ,βₘ=C_{e,m}fₛ, and kₒ is a time offset included to model potential temporal misalignment between measured ABP and CBF waveforms. In accordance with the model-imposed constraint of Equation 22, Equation 24 can be used,

\tilde{p}ᵢ[k]=γₘ\tilde{p}ₐ[k] (24)

where \tilde{p}ₐ is the pulsatile component of the ABP waveform, and γₘ is an unknown parameter.

Since all operations may be confined to individual data windows in the model above, the window index, m, is omitted for clarity in the remainder of this description of this non-limiting modeling approach. Estimates for α and β may be computed in a least-square-error sense for sets of the mean ICP, time offset, and γ

$${[ {\hat{α}}_{I , {k}_{o} , γ} , {\hat{β}}_{I , {k}_{o} , γ} ]}^{⊤} = {( {p}_{I , γ}^{⊤} {p}_{I , γ} )}^{†} {p}_{I , γ}^{⊤} {\dot{q}}_{{k}_{o}} where {\dot{q}}_{{k}_{o}} = {[ \dot{q} [ 2 - {k}_{o} ] , … , \dot{q} [ K - {k}_{o} ] ]}^{⊤} \qquad ( 25 )$$

$${p}_{I , γ} = [ \begin{matrix}( {p}_{a} [ 2 ] - γ {\tilde{p}}_{a} [ 2 ] - I & {p}_{a} [ 2 ] - {p}_{a} [ 1 ] \\ ⋮ & ⋮ \\ {p}_{a} [ K ] - γ {\tilde{p}}_{a} [ K ] - I & {p}_{a} [ K ] - {p}_{a} [ K - 1 ]\end{matrix} ]$$

Here, the dagger denotes a matrix pseudo-inverse, K denotes the number of samples in the estimation window, and l, kₒ, and γ signify the solution's dependence on the candidate mean ICP, time offset, and γ, respectively. Values for the cerebrovascular flow resistance and compliance may then be estimated according to

\hat{R}_{l,k}_{ₒ}_{,γ}=1/\hat{α}_{l,k}_{ₒ}_{,γ}

Ĉ_{e,l,k}_{ₒ}_{,γ}=\hat{β}_{l,k}_{ₒ}_{,γ}/fₛ (26)

The corresponding residual-error norm may be given by

ξ_{l,k}_{ₒ}_{,γ}=∥p_{l,γ}[\hat{α}_{l,k}_{ₒ}_{γ},\hat{β}_{l,k}_{ₒ}_{,γ}]^{T}−\dot{q}ₖ_{ₒ}∥₂ (27)

A likelihood distribution [symbol] (l, kₒ, γ) can be defined over the parameter search space

$$ℒ ( I , {k}_{o} , γ ) = \frac{exp \{ - {( \frac{{ξ}_{i , {k}_{o} , γ}}{{ξ}_{m}} )}^{2} \}}{{Z}_{ℒ}} \qquad ( 28 )$$

$${ξ}_{m} = {min}_{I , {k}_{o} , γ} {ξ}_{i , {k}_{o} , γ}$$

where [symbol] is an appropriate normalization constant. This formulation assigns a high likelihood to (l, kₒ, γ) sets that can result in a small residual CBF prediction error, and a low likelihood to sets with large residual error norms. [symbol] (l, kₒ, γ) may be marginalized across the time offsets and γ to generate a one-dimensional likelihood distribution defined across the mean ICP only

[symbol]

(l)=Σₖ_{ₒ}_{,γ} [symbol] (l,kₒ,γ) (29)

A mean ICP estimate may then be derived from the likelihood by computing a point statistic, Î_{L}, with an associated distribution variance,

$${σ}_{L}^{2} .$$

For this application, Î_{L}=median ( [symbol] (l)). An a posteriori distribution may also be generated by combining the likelihood distribution with a prior belief

$$Pr ( I \vert {p}_{a} , \dot{q} ) = \frac{Pr ( I ) ℒ ( I )}{{Z}_{P}} \qquad ( 30 )$$

where Pr(l) is the prior belief and Zₚ is a normalization constant. In some embodiments, a uniform prior belief is used. The resistance, effective compliance, and γ estimates may be determined by computing the appropriate a posteriori expectation. Then, using the relationship in Equation 22 along with the model of Equation. 24, estimates of C₁ and C₂ may be obtained according to

$${\hat{C}}_{1} = \frac{{\hat{C}}_{e}}{1 - \hat{γ}} , {\hat{C}}_{2} = \frac{{\hat{C}}_{e}}{\hat{γ}} \qquad ( 31 )$$

where \hat{γ} is the estimated value for the scaling constant. Possible time offsets may be selected on a window-by-window basis such that, on average, the CBF peaks are constrained to lead the corresponding ABP systolic peaks whilst ensuring that the diastolic points of the two waveforms are aligned with each other. These imposed constraints may be used to reflect the underlying Windkessel-like model that implies that both signals should start rising nearly simultaneously at the onset of systole, with the CBF rising faster to reach its peak before the ABP. To form the ICP scan range, scanning from an appropriate minimum value, Iₘᵢₙ, may be started in increments of 1 mmHg, a granularity generally deemed sufficient for clinical purposes. The scanning may be stopped at a preset maximum value, Iₘₐₓ. Data collected previously from Boston Children's Hospital has been used to determine an appropriate range for γ for some embodiments. Specifically, across 7 patients over 62 data recordings, the model of Equation 24 was fitted to measured ABP and invasively obtained ICP beats in a least-square-error sense. The resulting estimates of γ values had a mean of 0.12, and 25^{th} and 75^{th} percentiles of 0.06 and 0.18, respectively. Thus, γ was varied from 0.06 to 0.18 in increments of 0.01.

The approach described in this disclosure may be attractive for some applications, as it can be computationally simple and largely patient-specific. The model parameters are interpretable and may contribute to mechanistic insight into a subject's cerebrovascular physiology.

In some embodiments, the system comprises a display. Examples of connectivity to the display are described in more detail below. The display may be configured to communicate (e.g., to display) to a user (e.g., a healthcare provider) the information output from the at least one hardware processor (e.g., via displayed text, displayed graphics, and/or sound). In some embodiments, the display includes a graphical user interface (GUI). For example, the display may be configured to communicate (e.g., to display) to the user information indicating a measure of a value (e.g., in absolute units and/or in relative units) of intracranial pressure (ICP). As another example, the display may be configured to communicate (e.g., to display) to the user information indicating a measure of a value (e.g., in absolute units and/or in relative units) of intracranial compliance (ICC). As yet another example, the display may be configured to communicate (e.g., to display) to the user information indicating a measure of a value (e.g., in absolute units and/or in relative units) of cerebrovascular resistance (CVR). In some embodiments, the display is configured to communicate (e.g., to display) to the user information indicating a measure of a value (e.g., in absolute or relative units) one or more of the following: intracranial pressure (ICP), intracranial compliance (ICC), and cerebrovascular resistance (CVR). As noted above, in some instances it can be advantageous and an improvement over conventional technologies for the information output by the processor and communicated by the display to be in absolute units (e.g., units corresponding to pressure (force divided by area) for ICP, units corresponding to volume divided by pressure for ICC, units corresponding to pressure divided by a quantity of volume divided by time for CVR). The display of the information to the user may facilitate the user in monitoring and/or treating the subject (e.g., for a neuropathological condition).

In some embodiments, the display is configured to alert at least one user (e.g., a healthcare provider) based at least in part on the information output by processor. For example, in some embodiments, the instructions stored in the computer-readable storage medium may, when executed by the at least one hardware processor, further cause the hardware processor to generate an alert regarding the administration of a treatment for relieving a neuropathological or other condition based at least in part on the information output from the processor (e.g., indicating a measure of a value of ICP, ICC, and/or CVR). In some such embodiments, the instructions may further be configured to, when executed, cause the processor to transmit the alert to the display. The transmission of the alert to the display may be performed such that the alert triggers the display to communicate (e.g., display) the alert to at least one user (e.g., via text, graphics, and/or sound). In some embodiments, the processor is caused to prompt, using the alert generated by the processor and transmitted to the display, at least one user to administer the treatment to the subject. In some embodiments, the processor is caused to prompt, using the alert generated by the processor and transmitted to the display, the user(s) to begin, continue, and/or cease monitoring the subject. Such an alert may provide objective guidance to the user to care for a patient suffering and/or suspected of suffering from a neuropathological condition such as a traumatic brain injury. The alert may be generated and transmitted to the display when one or more of the measures of a value of ICP, ICC and/or CVR exceed or fall below a threshold (e.g., a predetermined threshold or a user-entered threshold).

In some embodiments, at least one user (e.g., a healthcare provider) can obtain a measure of intracranial pressure (e.g., in absolute and/or relative units) of a subject using the system described in this disclosure. The subject may be a patient (e.g., suffering from or suspected or suffering from a neuropathological condition such as traumatic brain injury). In some embodiments, the user can obtain a measure of intracranial compliance (e.g., in absolute and/or relative units) using the system described in this disclosure. In some embodiments, the user can obtain a measure of cerebrovascular resistance (e.g., in absolute and/or relative units) using the system described in this disclosure.

Some aspects of this disclosure relate to the administration of a treatment to a subject (e.g., a patient) based at least in part on a measure of one or more parameters (e.g., ICP, ICC, and/or CVR) obtained using the systems and methods described above. In some embodiments the subject is a human. In some embodiments, the subject is an animal (e.g., a non-human animal). For example, a user (e.g., a healthcare provider) may administer a treatment for relieving a neuropathological condition to a subject (e.g., a patient) based at least in part on the obtained measure. The neuropathological condition may be, for example, a traumatic brain injury (e.g., a severe traumatic brain injury) and/or a secondary injury following a traumatic brain injury (e.g., intracranial hypertension, brain swelling, reduced perfusion of surviving neural tissue, reduced oxygen and metabolite delivery, reduced clearance of metabolic waste and toxins). Other examples of neuropathological conditions that may be treated include, but are not limited to, hydrocephalus, hemorrhagic stroke, and/or brain tumors.

The administration of the treatment may be prompted and/or guided by the obtained measure. For example, as noted above, the system (e.g., via a display, which may include a GUI) may communicate (e.g., via text, graphic, and/or sound) an alert to the user (e.g., healthcare provider) to begin, modify, and/or cease administration of the treatment to the subject. In some embodiments, the user begins and/or continues monitoring of the one or more parameters (e.g., ICP, ICC, and/or CVR) at least in part based on the obtained information (e.g., due to an alert triggered by the information and communicated via, for example, a display of the system).

In some instances, the type, timing, and/or degree (e.g., dosage and/or duration) of treatment administered by the user is chosen based at least in part on the value of the obtained measure (e.g., of ICP, ICC, and/or CVR). For example, the type, timing, and/or degree of treatment may be based at least in part on whether the value of the obtained measure exceeds or is below a threshold (e.g., a predetermined threshold and/or a threshold entered by a user). For example, the treatment type, timing, and/or degree may be affected by whether the value of the obtained ICP exceeds or is below a value that is greater than or equal to 20 mmHg, greater than or equal to 22 mmHg, greater than or equal to 30 mmHg, greater than or equal to 35 mmHg, and/or up to 40 mmHg, up to 60 mmHg or greater. In some embodiments, the treatment type, timing, and/or degree may be affected by whether the value of the obtained ICP exceeds or is below 20 mmHg, 22 mmHg, 30 mmHg, 35 mmHg, 40 mmHg, or 60 mmHg.

In some embodiments, the administered treatment is a treatment for controlling intracranial hypertension. In some embodiments, the treatment is based on prevailing clinical guidelines for the management of severe traumatic brain injury for the relevant population for the treated subject (e.g., adult or pediatric). Examples of treatments (e.g., for a neuropathological condition such as traumatic brain injury) that may be administered based at least in part on the value of the obtained measure include, but are not limited to osmolar therapy (e.g., hyperosmolar therapy, such as a bolus of hypertonic saline (e.g., 3%) with dosage between 2-5 mL/kg over 10-2 min., or continuous infusion at dosage of between 0.1 and 1. mL/kg per hour), decompression (e.g., decompressive craniectomy), analgesics, sedatives, neuromuscular block, seizure prophylaxis, ventilation therapy (e.g., hyperventilation), temperature control, diuretics (e.g., mannitol, for example at doses of 0.25 to 1 g/kg body weight), cerebrospinal fluid (CSF) drainage, sedation, pharmacologic paralysis, and/or barbiturates. Further information of recommended treatments for severe brain injury (e.g., to control intracranial pressure) is provided in Carney, et al. (2017). “Guidelines for the management of severe traumatic brain injury.” Neurosurgery, 80 (1), 6-15, and in Kochanek, et al. (2019). “Guidelines for the management of pediatric severe traumatic brain injury: update of the brain trauma foundation guidelines.” Pediatric Critical Care Medicine, 20 (3S), S1-S82., each which is incorporated herein by reference.

An illustrative implementation of a computer system 1900 that may be used in connection with any of the embodiments of the technology described herein is shown in FIG. 4A. The computer system 1900 includes one or more processors 1910 and one or more articles of manufacture that comprise non-transitory computer-readable storage media (e.g., memory 1920 and one or more non-volatile storage media 1930). The processor 1910 may control writing data to and reading data from the memory 1920 and the non-volatile storage device 1930 in any suitable manner, as the aspects of the technology described herein are not limited in this respect. To perform any of the functionality described herein, the processor 1910 may execute one or more processor-executable instructions stored in one or more non-transitory computer-readable storage media (e.g., the memory 1920), which may serve as non-transitory computer-readable storage media storing processor-executable instructions for execution by the processor 1910.

Computing device 1900 may also include a network input/output (I/O) interface 1940 via which the computing device may communicate with other computing devices (e.g., over a network), and may also include one or more user I/O interfaces 1950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices. The display device may be the display described above (e.g., configured to communicate the information output from the processor).

As noted above, the system of this disclosure may comprise one or more probes (e.g., a single probe) configured to perform measurements at a patient site and send information about the measurements to the at least one hardware processor. FIG. 4B shows an illustrative implementation of one such embodiment, where system 10000 comprises computing device 1900 (described above) and probe 2000 configured to interface with computing device 1900. Probe 2000 can be, for example, an ultrasound probe. In some embodiments, probe 2000 comprises one or more sensors. The sensors may include ultrasonic transducers (e.g., capacitive micromachined ultrasonic transducers (CMUTs) or piezoelectric ultrasonic transducers, among other ultrasonic transducers), and/or sensors other than ultrasonic transducers.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

U.S. Provisional Patent Application No. 63/413,875, filed Oct. 6, 2022, and entitled “System and Methods for Measurement of Parameters such as Intracranial Pressure,” is incorporated herein by reference in its entirety for all purposes.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system comprising:
at least one hardware processor; and
at least one non-transitory computer-readable storage medium storing processor-executable instructions that, when executed by the at least one hardware processor, cause the at least one hardware processor to perform:
obtaining a set of data identifying a measure of arterial blood pressure and a measure of volumetric cerebral blood flow of a patient; and
outputting information, based at least in part on the set of data, indicating a measure of a value in absolute units of one or more of the following: intracranial pressure (ICP), intracranial compliance (ICC), and cerebrovascular resistance (CVR).

2. The system of claim 1, further comprising one or more probes configured to perform measurements at a patient site and send information about the measurements to the at least one hardware processor, wherein the obtaining the set of data identifying a measure of arterial blood pressure and a measure of volumetric cerebral blood flow of a patient is based at least in part on the information about the measurements at the patient site.

3. The system of claim 1, wherein the measure of a value in absolute units of ICC is a measure of a value in absolute units of a regional ICC and/or wherein the measure of a value in absolute units of CVR is a measure of a value in absolute units of a regional CVR.

4. A system comprising:
at least one hardware processor;
one or more probes configured to perform measurements at a patient site and send information about the measurements to the at least one hardware processor; and
at least one non-transitory computer-readable storage medium storing processor-executable instructions that, when executed by the at least one hardware processor, cause the at least one hardware processor to perform:
obtaining a set of data, based at least in part on the information about the measurements at the patient site, identifying measures of two or more parameters related to distension of a vessel and blood flow through the vessel; and
outputting information, based at least in part on the set of data, indicating a value of one or more of the following: intracranial pressure (ICP), intracranial compliance (ICC), and cerebrovascular resistance (CVR).

5. The system of claim 4, wherein the value indicated in the output information is a value in absolute units.

6. The system of claim 4, wherein the measures of two or more parameters related to distension of a vessel and blood flow through the vessel comprise one or both of:
(1) a measure of arterial blood pressure; and
(2) a measure of cerebral blood flow.

7. The system of claim 6, wherein the measure of cerebral blood flow comprises a measure of cerebral blood flow velocity and/or a measure of volumetric cerebral blood flow.

8. The system of claim 2, wherein the one or more probes comprise only a single probe.

9. The system of claim 2, wherein the one or more probes comprise an ultrasound probe.

10. The system of claim 1, wherein the information output by the at least one hardware processor indicates a measure of a value in absolute units of ICP.

11. The system of claim 10, wherein the measure of a value in absolute units of ICP is in the units of mmHg, pascals, bars, pounds per square inch (PSI), atmospheres, or a multiple thereof.

12. The system of claim 1, wherein the information output by the at least one hardware processor indicates a measure of a value in absolute units of ICC.

13. The system of claim 12, wherein the measure of a value in absolute units of ICC is in the units of mL/mmHg, mL/pascal, mL/bar, mL/PSI, mL/atmosphere, or a multiple thereof.

14. The system of claim 1, wherein:
(a) the data identifying a measure of arterial blood pressure comprise arterial blood pressure waveforms converted from vessel diameter waveforms using pre-trained models, first principle models, and/or by calibration with an arm-cuff; and/or
(b) the data identifying a measure of volumetric cerebral blood flow of a patient are calculated via integration of a measure of cerebral blood flow velocity using vessel diameter waveforms; and/or
(c) the data identifying a measure of arterial blood pressure and/or the data identifying a measure of cerebral blood flow are obtained based at least in part on information about measurements from a biplanar imaging sequence.

15. The system of claim 1, wherein the values of ICP, ICC, and/or CVR are calculated by feeding arterial blood pressure and volumetric cerebral blood flow waveforms to a modified physiologic model.

16. A point-of-care system configured to perform a continuous, model-based, non-invasive, real-time, patient-specific measurement of a measure of intracranial pressure (ICP), intracranial compliance (ICC), and/or cerebrovascular resistance (CVR).

17. The system of claim 1, further comprising a display configured to communicate to at least one user the information output from the at least one hardware processor.

18. The system of claim 17, wherein the instructions, when executed by the at least one hardware processor, further cause the at least one hardware processor to perform:
generating an alert regarding the administration of a treatment for relieving a neuropathological condition based at least in part on the information output from the at least one hardware processor; and
transmitting the alert to the display such that the alert triggers the display to communicate the alert to the at least one user.

19. A method, comprising obtaining a measure of one or more of the following using the system of claim 1: intracranial pressure (ICP), intracranial compliance (ICC), and cerebrovascular resistance (CVR).

20. The method of claim 19, further comprising administering a treatment for relieving a neuropathological condition to a subject based at least in part on the obtained measure.
